(19) United States
(12) Patent Application Publication
(10) Pub. No.: US 2026/0232893 A1
(43) Pub. Date: Aug. 13, 2026

(54) AUTOMATED MEDICAMENT DELIVERY WITH UNLOCKABLE FEATURES AND FUNCTIONS

(71) Applicant: Insulet Corporation, Acton, MA (US)

(72) Inventors: John D'Arco, Wilmington, MA (US); Joon Bok Lee, Acton, MA (US); Stephen Baumhover, Acton, MA (US); William J. Whiteley, Knoxville, TN (US); Jason O'Connor, Acton, MA (US)

(21) Appl. No.: 19/535,964

(22) Filed: Feb. 10, 2026

Related U.S. Application Data
(60) Provisional application No. 63/756,538, filed on Feb. 10, 2025.

(51) Int. Cl. A61M 5/142

(52) U.S. Cl. CPC A61M 5/142; A61M 2005/14208; A61M 2202/0007; A61M 2202/04; A61M 2205/18; A61M 2205/502

(57) ABSTRACT

A method of automated delivery of medicament is disclosed that comprises operating an automated medicament delivery device in a default mode and/or user interface, wherein at least one operating parameter of the device is set to a preset value to ensure safe delivery of the medicament. The method comprises setting a condition for a threshold value for at least one selected threshold parameter and providing a user of the device with the capability option to manually adjust or control at least one operating parameter for an administration of the medicament once the threshold value for the at least one threshold parameter is met.

### CROSS-REFERENCE TO RELATED APPLICATIONS

This application claims the benefit under 35 U.S.C. § 119(e) of U.S. Provisional Patent Application Ser. No. 63/756,538, filed Feb. 10, 2025, the disclosure of which is hereby incorporated herein in its entirety by this reference.

### FIELD

The present disclosure generally relates to medicament delivery devices and systems. More particularly, the present disclosure relates to automated medicament delivery devices and systems with unlockable progressive features, and related methods for automated delivery of medicament.

### BACKGROUND

Automated medicament delivery devices (e.g., an automated insulin delivery device, without limitation) are often used to administer medicament to the body of a patient via a transfer mechanism to treat medical conditions. However, the complexity of the user interface modes of such a device is a large concern for the users, including users who desire to change from a manual medicament delivery device to an automated medicament delivery device (e.g., those practicing multiple daily Injection considering switching to using an automated insulin delivery (AID) device, without limitation). Although efforts have been made to simplify the user interface modes, the fundamental need to enter a wide range of parameters and settings to begin utilization of an automated medicament device remains a difficult hurdle when reducing the complexity of the user interface modes.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a schematic diagram illustrating a device or system for automated administration of medicament to a user-body, in accordance with one or more embodiments.

FIG. 2 is a block diagram of an automated medicament delivery system for controlled administration of medicament to a user-body, in accordance with one or more embodiments of present disclosure.

FIG. 3 is a flow diagram depicting a process progressively unlocking features and functions of an automated medicament delivery system as conditions are met, in accordance with one or more embodiments.

FIG. 4 is a flow diagram illustrating a process to initially configure an automated medicament delivery system, in accordance with one or more embodiments.

FIG. 5 is a flow diagram depicting a process for progressive unlocking of various aspects of automated medicament delivery, in accordance with one or more embodiments.

FIG. 6 shows non-limiting examples of the preset values for different operating parameters when the automated medicament delivery device (e.g., AID device equipped with insulin pump, without limitation) is operated in a default mode.

FIG. 7 is a table of non-limiting examples of threshold parameters that may be utilized to adapt a delivery mode and/or UX, according to one or more embodiments of present disclosure.

FIG. 8 provides non-limiting examples of unlockable features of an automated medicament delivery system, according to one or more examples.

FIG. 9 provides non-limiting examples of features or functions of an automated medicament delivery system available for unlocking, according to one or more examples.

FIG. 10 is a flow diagram of a condition processing chain, in accordance with one or more embodiments.

FIG. 11 is a flow diagram of a process for automated delivery of medicament in accordance with one or more embodiments. FIG. 11 depicts a specific use case where the A1C value is used as the threshold parameter.

FIG. 12 is a flow diagram of a process for automated delivery of medicament in accordance with one or more embodiments, wherein the duration of using the automated medicament delivery device is used as the threshold parameter.

FIG. 13 is a flow diagram of a process for automated delivery of medicament in accordance with one or more embodiments, wherein a goal set by the user of the automated medicament delivery device is used as the basis for the threshold parameter and threshold value.

FIG. 14 is a table showing non-limiting examples of the combinations of features in the delivery mode and/or user interface that may be progressively unlocked as the user achieves different levels of A1C values, across feature categories including overall use complexity, alerts and reminders, application or personal diabetes management (PDM) use, meal announcements, food and correction bolus, bolus calculator, basal profile, activity mode, and AID algorithm auto-adjustments, according to one or more embodiments.

FIG. 15 is a flow diagram of a process for automated delivery of medicament in accordance with one or more embodiments, wherein the automated medicament delivery device monitors whether a threshold value of a selected threshold parameter continues to be fulfilled after features or functions have been unlocked, and reverses the operation back to default features or functions in a default mode if the threshold value is no longer met.

### DETAILED DESCRIPTION

In various embodiments, the automated medicament delivery devices with unlockable progressive features, as well as the methods for automated delivery of medicament using such devices are disclosed.

In the following detailed description, reference is made to the accompanying drawings, which form a part hereof, and in which are shown, by way of illustration, specific examples of embodiments in which the present disclosure may be practiced. These embodiments are described in sufficient detail to enable a person of ordinary skill in the art to practice the present disclosure. However, other embodiments may be utilized, and structural, material, and process changes may be made without departing from the scope of the disclosure.

The illustrations presented herein are not meant to be actual views of any particular method, system, device, or structure, but are merely idealized representations that are employed to describe the embodiments of this disclosure.

The following description may include examples to help enable one of ordinary skill in the art to practice the disclosed embodiments. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

The embodiments may be described in terms of a process that is depicted as a flow diagram, swim-lane diagram, a structure diagram, or a block diagram. Although a flow diagram may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, any relational term, such as “first,” “second,” “top,” “bottom,” “upper,” “lower,” “above,” “beneath,” “side,” “upward,” “downward,” etc., is used for clarity and convenience in understanding the disclosure and accompanying drawings, and does not connote or depend on any specific preference or order, except where the context clearly indicates otherwise. For example, these terms may refer to an orientation of elements of any system, device, or structure, when utilized in a conventional manner. Furthermore, these terms may refer to an orientation of elements of any system, device, or structure, as illustrated in the drawings.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, without limitation).

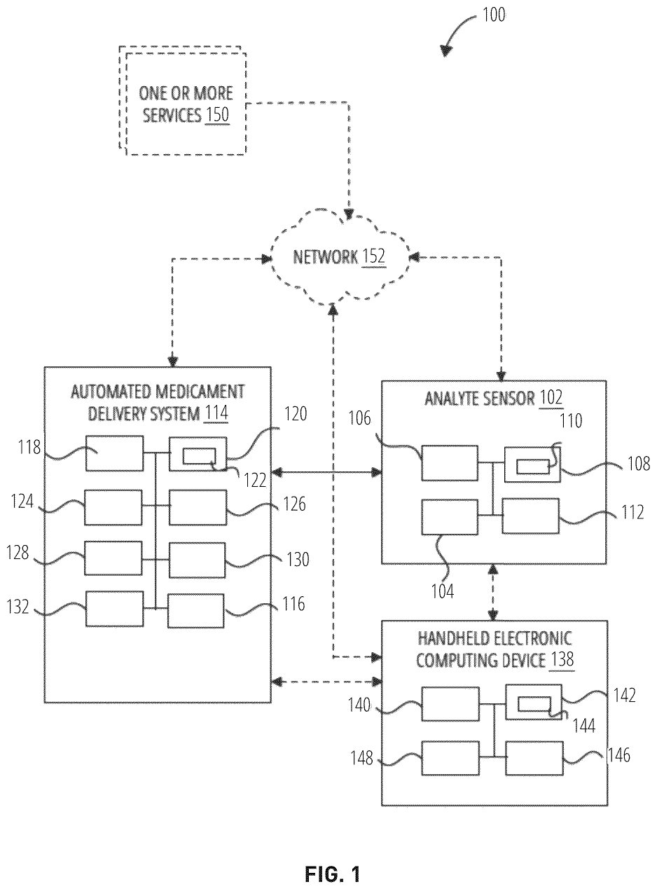

FIG. 1 is a schematic diagram illustrating a device or system 100 (system 100) for automated administration of medicament to a user-body, in accordance with one or more embodiments.

In one or more embodiments, system 100 may be capable of one or more modes of operations of administration of medicament (“delivery modes”). Non-limiting examples of the one or more modes of operations include: fully automated administration of medicament, partially automated administration of medicament, or manual administration of medicament. In one or more embodiments, the system 100 may be capable of changing between multiple (e.g., two or more, without limitation) modes of operations. As a non-limiting example, the system 100 may at least change between one or more of: fully automated operation, partially automated operation, and manual operation.

System 100 may administer medicament at least partially based on one or more values representative of amounts of one or more analytes present within a user-body (such values respectively an “analyte value”). The one or more analytes may include constituents of the user-body and foreign substances, such as medicaments, markers, metabolites, and any combination thereof, without limitation. Analyte values may include values representative of amounts of one or more analytes present within a user-body and values at least partially based on the same such as the A1C value, the blood glucose value (e.g., milligrams per deciliter (mg/dL), without limitation), the insulin-to-carbohydrate (I:C) ratio, or any combination thereof, without limitation.

Non-limiting examples of medicaments administrable by the system 100 include: insulin, glucagon-like peptide-1 receptor agonist (GLP-1), glucose-dependent insulinotropic polypeptide (GIP), insulin substitute, hormone, or any combination thereof, such as two or more of insulin, GLP-1, and GIP, or other like hormones. While specific examples discussed herein may involve insulin, this disclosure is not limited to those examples, and other medicaments do not exceed the scope of this disclosure. As a non-limiting example, glucagon, morphine, analgesics, fertility medicaments, blood pressure medicaments, chemotherapy drugs, arthritis drugs, weight loss drugs, without limitation are non-limiting examples of medicaments that are specifically contemplated.

System 100 includes an analyte sensor 102 and an automated medicament delivery system 114. The system 100 may optionally include a handheld electronic computing device 138, a network 152, and a service 150.

The analyte sensor 102 is configured to obtain data related to one or more analytes within the user-body (“analyte data”). In various embodiments, the analyte data may include one or more analyte values. In various embodiments, the analyte sensor 102 is an analytical bio-sensing device, such as a continuous glucose monitor (CGM) or an integrated continuous glucose monitor (ICGM) (e.g., examples of commercially available analytical bio-sensing devices include the FREESTYLE LIBRE® 3 manufactured by Abbott or the DEXCOM® G6 manufactured by Dexcom, without limitation).

The analyte sensor 102 includes a filament 104 and various electronic components. The filament 104 is configured to obtain data related to one or more analytes within a user-body and provide the data to the various electronic components of the analyte sensor 102. The filament 104 may be configured to obtain the data directly from fluids of a user-body, including without limitation interstitial fluids of a user-body, from tissue of a user-body, any combination thereof, or in any other manner known in the art.

The various electronic components of analyte sensor 102 include a processor 106, a memory 108, and a communication equipment 112. Instructions 110 include instructions for processing data obtained via the filament 104. When the instructions 110 are executed by the processor 106, the instructions 110 cause the processor 106 to process the data obtained via the filament 104. The instructions 110 may be implemented in hardware (e.g., one or more hardware processors of the processor 106 such as an integrated circuit, application specific integrated circuit (ASIC), digital signal processor (DSP), or other logic circuit, without limitation), implemented in software (e.g., firmware, software, machine code, applications, without limitation), or any combination thereof. The instructions for processing the data obtained via the filament 104 may include one or more instructions respectively for determining analyte values at least partially based on the data, or for sending the data, analyte values or both to the automated medicament delivery system 114 or the service 150.

The communication equipment 112 is configured to facilitate communication (e.g., a device or interface for wired communication, wireless communication, both wired and wireless communication, without limitation) of the analyte sensor 102 with other components of the device, including the automated medicament delivery system 114, without limitation. Such communication may be according to any appropriate wired or wireless communication protocol, such as WI-FI®, BLUETOOTH®, near-field communication (NFC), radio-frequency identification (RFID), or any other radio-frequency, infrared or optical communication technology.

The automated medicament delivery system 114 is configured to administer medicament to a user-body, such as subcutaneously into the user-body, without limitation, in accordance with one or more embodiments. In one or more embodiments, the automated medicament delivery system 114 may offer one or more modes of operations for administration of medicament to a user-body. When operating in some of the modes of operations, the automated medicament delivery system 114 may administer medicament at least partially responsive to analyte values (e.g., partially responsive to or solely responsive to, without limitation), including without limitation analyte values received from the analyte sensor 102. When operating in some further modes of operations, the automated medicament delivery system 114 may administer medicament at least partially responsive to user input. When operating in yet some further modes of operation, the automated medicament delivery system 114 may administer medicament at least partially responsive to analyte values and user input. Non-limiting examples of the one or more modes of operations offered by the automated medicament delivery system 114 include: fully automated administration of medicament, partially automated administration of medicament, or manual administration of medicament. When operating in the various modes of operation that includes manual administration of medicament, the automated medicament delivery system 114 may administer medicament solely in response to a user input (e.g., delivers medicament in response to a user confirmation of delivery of medicament or in response to a user instruction to delivery medicament, without limitation). When operating in the modes of operation that includes fully automated administration of medicament, the automated medicament delivery system 114 may administer medicament solely in response to analyte values (e.g., delivers medicament in response to one or more analyte values, without limitation). When operating in the modes of operation that includes partially automated administration of medicament, the automated medicament delivery system 114 may administer medicament in response to analyte values and user input (e.g., delivers medicament in response to a user input and an analyte value, or alternately delivers medicament in response to a user input or in response to analyte values, without limitation).

In one or more embodiments, automated medicament delivery system 114 may include adaptive control that changes (at least temporarily) aspects of delivery modes, user interface (UI), or user experience (UX) (UI and UX are collectively referred to herein as “UX” unless otherwise stated) for a user based on one or more predetermined conditions, as discussed below. Such adaptive control may be implemented by logic (“adaptive control logic”) of, as non-limiting examples, automated medicament delivery system 114, a handheld electronic computing device 138, or both.

In one or more embodiments, the automated medicament delivery system 114 may include a delivery unit 116, a processor 118, a memory 120, a communication equipment 124, a power source 126, and optionally a sensor 128. In one or more embodiments, the automated medicament delivery system 114 or portions thereof, may be a wearable device and may be secured to a user-body (e.g., secured via one or more adhesive layers attaching the automated medicament delivery system 114 to the skin of the user-body or a material that is secured to the user body, without limitation).

In various embodiments, the delivery unit 116 of the automated medicament delivery system 114 is configured to cause an amount of medicament to move (e.g., flow, without limitation) toward and/or into a user-body.

In various embodiments, the delivery unit 116 of the automated medicament delivery system 114 may deliver amounts of medicament at least partially responsive to requests. In various embodiments, the instructions 122 of the memory 120 may include instructions for determining and generating requests for the delivery unit 116. In various embodiments, the instructions 122 may include instructions for determining one or more amounts of medicament, determining a timing for delivery of one or more amounts of medicament, and for generating one or more requests for the delivery unit 116 related to the same. When such instructions of the instructions 122 are executed by the processor 118, the processor 118 may determine the amounts of medicament and timing of delivery, generate requests for the delivery unit 116 at least partially based on the determined amounts and timing, and provide the requests to the delivery unit 116.

The communication equipment 124 is configured to facilitate communication (e.g., wireless communication, without limitation) of the automated medicament delivery system 114 with other components of the automated medicament delivery device, including without limitation communication between the analyte sensor 102 and the automated medicament delivery system 114. The communication may be wired or wireless communication and may utilize any suitable communication protocol such as wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In various embodiments, the communication equipment 124 includes an Internet of Things (IOT) Subscriber Identity Module (SIM) card (e.g., a machine-to-machine SIM card, a Universal Integrated Circuit Card, without limitation).

The power source 126 is configured to supply power to the delivery unit 116 and the various electronic components of the automated medicament delivery system 114, such as the processor 118, the memory 120, the communication equipment 124, and the like. The power source 126 may be, as a non-limiting example, a power storage device (e.g., a battery, without limitation), a power inlet, a power regulator, or combination thereof.

In various embodiments, the automated medicament delivery system 114 may include one or more of: a sensor 128, an input device 130, and an output device 132. The sensor 128 may include a sensor (i.e., one or more sensors chosen from among an accelerometer, a gyroscope, and an inertial measurement unit (IMU) or similar sensors). The sensor unit (i.e., one or more sensor units) 128 may further include a data sensor unit (i.e., one or more data sensor units). Such data sensor unit may include a sensor unit (i.e., one or more sensor units) chosen from a geographic sensor (e.g., a sensor for the Global Positioning System (GPS) operated by the United States Department of Defense, without limitation), an electrocardiogram sensor, a heart rate sensor, a skin conductance sensor, and the like. In one or more embodiments, the sensor 128 or automated medicament delivery system 114 more generally, may be configured to relate to one or more of a geographic location of the automated medicament delivery system 114, data regarding operation of the automated medicament delivery system 114, and patient data (e.g., electrical activity of the heart, heart rate, skin conductance, without limitation). One or more sensors 128 may include physical attribute sensors such as weight sensors, heart-rate sensors, temperature sensors, without limitation.

In one or more embodiments, the automated medicament delivery system 114 may be configured to utilize information provided by the sensor 128 to determine whether the automated medicament delivery system 114 is positioned on a user-body (“on-body sensor”), is operating correctly, or is operating in suitable environment, without limitation.

In one or more embodiments, the automated medicament delivery system 114 may be configured to utilize information (e.g., analyte values, without limitation) provided by the communication equipment 112 of the analyte sensor 102 and administer medicament at least partially responsive to information (e.g., analyte values, without limitation).

In one or more embodiments, the automated medicament delivery system 114 may be configured to utilize information (e.g., activity level, stress level, wellness level, meal announcement, without limitation) provided by the user via the input device 130 of the automated medicament delivery system 114 and administer medicament at least partially responsive to information provided by the user, such as, without limitation, when the automated medicament delivery system 114 is operated.

The input device 130 may include a button (e.g., a mechanical button, capacitive button, a touch screen button, without limitation), a touch screen device, and/or a microphone configured to receive an input from the user (e.g., a button press, a tap, and/or a sound, without limitation).

The output device 132 may include one or more of: a display, an audio speaker, a Light Emitting Diode (LED), and/or vibration motor (e.g., a vibration transducer, without limitation) configured to provide a signal to the user (e.g., a sound, a visual signal, such as light, and/or vibration, without limitation). The audio speaker may be configured to play multiple tones with various patterns. The processor 118 may utilize a message parser to determine the message type, refer processes associated with medicament delivery to a main process or a main processor of the one or more processors 118, and refer special alert messages on to a message generator (e.g., a tone and pattern generator which drives the audio speaker or a vibration generator which drives the vibration motor, without limitation).

In various embodiments, the communication equipment 124 of the automated medicament delivery system 114 and the communication equipment 112 of the analyte sensor 102 may be configured to communicate with the service 150 via network 152. In various embodiments, the service 150 may be provided by one or more computer servers or cloud computer servers. A respective server may be a dedicated secure endpoint configured to communicate with the automated medicament delivery system 114 and/or the analyte sensor 102. In various embodiments, the communication equipment 124 of the automated medicament delivery system 114 and the communication equipment 112 of the analyte sensor 102 may be configured to communicate via a short-range wireless protocol, and in response to the inability to communicate via the short-range wireless protocol, communicate via the service 150 (e.g., the automated medicament delivery system 114 utilizes a IOT SIM card to communicate with the one or more services 150 to send and receive messages to and from the handheld electronic computing device 138).

In various embodiments, the handheld electronic computing device 138 is configured to communicate with the automated medicament delivery system 114 and the analyte sensor 102. The handheld electronic computing device 138 may be chosen from among a dedicated electronic device, a smart phone, a tablet computer, a wearable device (e.g., a smart watch, without limitation), a cloud computing device, and the like.

The handheld electronic computing device 138 may include a processor (i.e., one or more processors) 140, a memory 142 that stores instructions 144 to be executed by the processor 140, a communication equipment 146, and a user interface 148. The processor 140 and the memory 142 may be configured/programmed to perform any of the operations discussed above, as well as other control operations for managing the automated medicament delivery system 114 and the analyte sensor 102.

The communication equipment 146 is configured to facilitate communication (e.g., wireless communication, without limitation) of the handheld electronic computing devices 138 with other devices, such as the automated medicament delivery system 114, the analyte sensor 102, and the network 152. The communication may be wired or wireless communication, such as via a wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In some of these embodiments, the automated medicament delivery system 114 and the handheld electronic computing device 138 are paired via the short-range wireless protocol (e.g., paired via BLUETOOTH®, without limitation) and successful message transmissions between the automated medicament delivery system 114 and the handheld electronic computing devices 138 may be acknowledged.

In various embodiments, the communication equipment 124 of the automated medicament delivery system 114 and the communication equipment 146 of the handheld electronic computing device 138 are configured to communicate with and via a service 150 (i.e., one or more services). The service 150 may be a dedicated secure endpoint configured to communicate with the handheld electronic computing device 138 and other similar devices. In some of these various embodiments, the communication equipment 124 of the automated medicament delivery system 114 and the communication equipment 146 of the handheld electronic computing device 138 are configured to communicate via the short-range wireless protocol, and in response to the inability to communicate via the short-range wireless protocol, communicate via the one or more services 150 (e.g., the automated medicament delivery system 114 utilizes a IOT SIM card to communicate with the one or more services 150 to send and receive messages to and from the handheld electronic computing device 138).

The user interface 148 is configured to provide a user with information and obtain information from the user via one or more of a display, an audio speaker, an LED, a vibration motor, a button (e.g., a mechanical button, capacitive button, without limitation), a gesture-based interface, and the like.

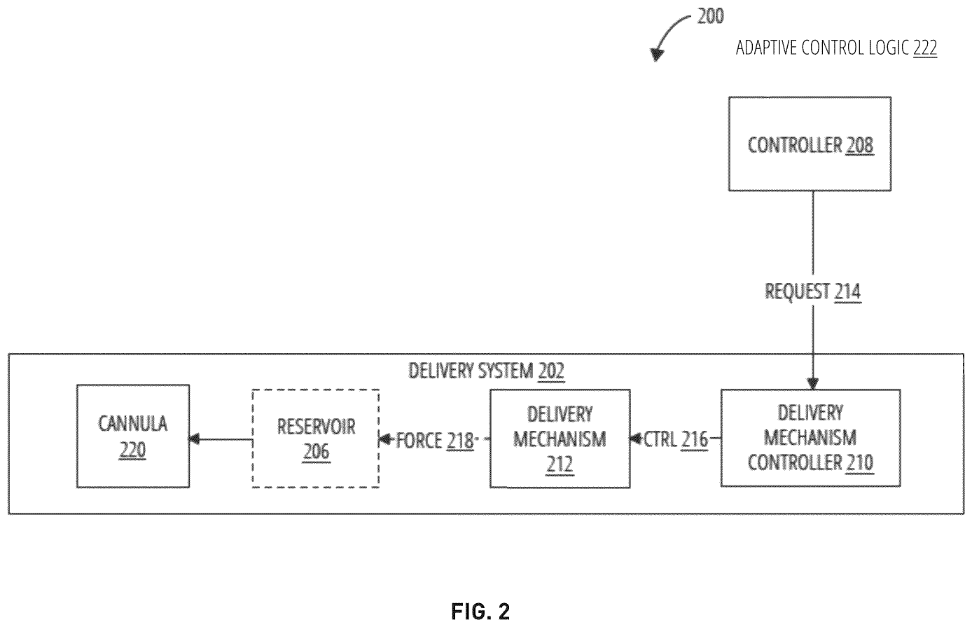

FIG. 2 is a block diagram of an automated medicament delivery system 200 for controlled administration of medicament to a user-body, in accordance with one or more embodiments of present disclosure.

The controller 208 is configured to manage automated medicament delivery system 114 of the system 100 shown in FIG. 1 and, more generally, administration of medicament to a user-body. In one or more embodiments, the controller 208 may be implemented by the instructions 122 and the processor 118 of the automated medicament delivery system 114 of FIG. 1.

In various embodiments, the controller 208 and the delivery system 202 may be realized in different devices (e.g., the controller 208 may be realized in a physically different device from the delivery system 202, without limitation) or in the same device. When realized in different devices, functionality of the controller 208 and the delivery system 202 may be implemented, at least in part, by respective memory and one or more processors of their respective devices. When realized in a same device, functionality of the controller 208 and the delivery system 202 may be implemented, at least in part, by like memory and like processor, respective memory and respective processor, or any combination thereof. Non-limiting examples of devices in which the controller 208 or a portion thereof, may be realized include: a handheld electronic computing device, such as a dedicated electronic device, a smart phone, a tablet computer, a wearable device (e.g., a smart watch, without limitation), a cloud computing device, and the like.

In various embodiments, the controller 208 may be configured to receive analyte data including analyte values (e.g., from the analyte sensor 102, from one or more services 150, or both, without limitation). In one or more embodiments, the controller 208 may determine information about analytes within a user-body at least partially based on the analyte data, for example, amounts, trends, distributions, without limitation. The controller 208 may analyze information about analytes in a user-body and may present the information and/or analysis to a patient (e.g., the user of the medicament delivery system), a caregiver, or a healthcare provider, as a non-limiting example, via the output device 132 of FIG. 1 or via an application (e.g., executing on a personal computer, smart phone, cloud server, or any combination thereof).

In various embodiments, the controller 208 may be configured to receive information from the analyte sensors, the inputs from the patient or the caregiver (e.g., when the patient ate a meal or when the patient exercised, without limitation), and the inputs from other electronic devices (e.g., information from a smartwatch, without limitation), as well as to utilize such information as discussed herein. For example, in various embodiments, the controller 208 may utilize some or a totality of such information to determine amounts of medicament to administer and timing of administration of medicament. Further, the controller 208 may also be configured to determine requests, including a request to administer dose 214, and send those requests to the automated medicament delivery system 114.

In various embodiments, the controller 208 may be configured to determine a target dose amount to administer to a user of automated medicament delivery system 200. The controller 208 may determine a target dose amount at least partially based on therapy parameters, meal information, analyte values, and a control algorithm, without limitation.

In the context of insulin therapy to treat diabetes, therapy parameters may include insulin sensitivity factor (ISF), carbohydrate ratio (CR), amount of daily dose of long-acting insulin (LAI), a current glucose value, and derivatives thereof without limitation. The timing and target dose amounts associated with requests generated by the controller 208 may be governed by one or more control algorithms.

The controller 208 may send a request to administer dose to the delivery system 202, and more specifically, the delivery mechanism controller 210. The request to administer dose may include the target dose amount determined by the controller 208.

In one or more embodiments, controller 208 (or automated medicament delivery system 114 more generally) may include an adaptive control logic 222 configured to change (at least temporarily) aspects of delivery modes and/or UX for a user based on one or more conditions. Although adaptive control logic 222 is depicted at controller 208, some or a totality of features or functions attributed herein to adaptive control logic 222 may be partitioned and located in a variety of ways and not required to be entirely located at one or more of: controller 208. As non-limiting examples, various logic portions of adaptive control logic 222 may be located at controller 208, handheld electronic computing device 138, or one or more services 150.

In one or more embodiments, controller 208 may start with an initial configuration that sets the delivery mode, UX or both. Adaptive control logic 222 may change aspects of the initial configuration in response to predetermined conditions. By way of a specific, non-limiting example of an initial configuration, the delivery mode of automated medicament delivery system 114 is set to an automated basal mode, and the UX is set to a minimal UI that facilitates input of a predetermined set of parameters. The predetermined set of parameters may correspond to a fewest number of parameters required to ensure safe operation in an automated basal mode.

In an automated basal mode, controller 208 automatically administers basal amounts of medicament without user input and, optionally, without analyte values. Automated basal mode seeks to maintain a steady level or delivery rate of medicament based on basal (e.g., background, without limitation) needs of a user. Automated basal mode may be appropriate for, as non-limiting examples, new users or users unfamiliar with, or overwhelmed by, more complex features (i.e., more complex than the features offered during automated basal mode).

Upon starting in the initial configuration, the adaptive control logic may adapt aspects of the delivery mode and/or UX based on conditions. While non-limiting examples of adaptations and conditions are provided herein, specific adaptations and conditions may be at least partially based on specific operating conditions.

The cannula 220 is insertable into a user-body (e.g., with a tip thereof positioned subcutaneously, without limitation) and is configured to provide medicament to the user-body (e.g., subcutaneously into the user-body, without limitation).

The reservoir 206 is configured to store and retain a medicament therein. As a non-limiting example, the reservoir 206 may be a hollow body, a chamber, a vial, without limitation. In various embodiments, the reservoir 206 is a fluid reservoir for holding medicament and may be, as a non-limiting example, formed from the walls of a cartridge. In the cartridge example, the delivery system 202 may include a chamber (i.e., a space or region defined within the delivery system 202) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in the reservoir 206 will be greater in a pre-filled state than the volume in an exhausted state. Additionally or alternatively to the cartridge example, the delivery system 202 is a multi-part delivery device where one of the two parts includes the reservoir 206 and the other one of the two parts includes the delivery mechanism controller 210. The other one of the two parts may optionally further include a controller 208. Either one of the two parts may optionally include a delivery mechanism 212 (e.g., a pump mechanism, without limitation). The one of the two parts that includes the reservoir 206 is disposable (i.e., a “disposable part”) and configured to be removable secured to the other part of the automated medicament delivery system 200. When the reservoir 206 is exhausted, the disposable part may be removed and a replacement part including a reservoir 206 optionally in a pre-filled state.

The delivery mechanism 212 is configured to urge fluid in the reservoir 206 toward an interface for dispensing fluid (interface not shown). In various embodiments, the delivery mechanism 212 may be positioned adjacent to the reservoir 206. The delivery mechanism 212 is configured to cause an amount of the medicament to be administered to the user-body by causing the amount to flow from the reservoir 206 toward and into a user-body via the cannula 220, which is in fluidic communication with the reservoir 206. In various embodiments, the delivery mechanism 212 may utilize any suitable mechanism to generate positive displacement or negative displacement to transfer amounts of medicament from the reservoir 206 toward the cannula 220 and the user-body. Non-limiting examples of mechanisms include a ratchet gear pump, peristaltic pump, linear peristaltic pump, piston pump, gear pump, bellows pump, or diaphragm pump.

For example, the delivery mechanism 212 may apply a force to an urging mechanism (e.g., a plunger, flexible-walled tube, without limitation) free to move within the reservoir 206, and via such a force, move the urging mechanism in a direction that urges fluid in the reservoir 206 toward the aforementioned interface. In one or more examples, the delivery mechanism 212 may include an electrical motor (e.g., an AC or DC motor) that produces a force to, directly or indirectly, move the urging mechanism to perform a delivery action. The delivery action dispenses the medicament at a predetermined rate (i.e., a predictable amount of fluid over a predictable duration of time). The delivery mechanism 212 may be capable of multiple rates of delivery, and in one or more examples, may be preconfigured to use a same rate of delivery all the time, or, in some cases, may be provided discretion to determine a rate of delivery consistent with a target dose amount included with a request.

Such an electric motor may be a current controlled electric motor, voltage controlled electric motor, pulse-width controlled electric motor, or combination or sub combination thereof. Such an electronic motor may be directly or indirectly digitally controlled. The control signal 216 may be determined and generated by the delivery mechanism controller 210 corresponding to a delivery action. A control signal 216 may also be referred to herein as a “command 216” or an “instruction 216.”

The delivery mechanism controller 210 may generate the control signals 216 corresponding to one or more delivery actions at least partially based on the request to administer dose 214 received from the controller 208. The control signal 216 may include the first control signals to cause the delivery mechanism 212 to generate a resultant force 218, and the second different control signals to cause the delivery mechanism 212 to stop generating or to not generating the force 218. Utilizing the control signals 216, the delivery mechanism controller 210 may control a length of a duration of time that the delivery mechanism 212 produces the force 218 and applies it to dispense fluid from the reservoir 206, and indirectly, an amount of fluid dispensed from the reservoir 206.

When the delivery mechanism controller 210 generates the control signal 216 in response to the request to administer dose 214 from the controller 208, it may generate the control signal 216 at least partially based on a value of a target dose amount included with, or indicated by, the request to administer dose 214. One or more delivery actions may be utilized to dispense an amount fluid corresponding to a dose amount determined by the controller 208. For example, the fluid amount dispensed according to a delivery action may be less than a dose amount. Generally speaking, the delivery mechanism 212, and the delivery system 202, are agnostic to the purpose for which fluid is dispensed and unaware of what constitutes a working amount of fluid to administer a dose, or series of doses, of medicament. So, while it may be desirable that a fluid amount dispensed according to one or more delivery actions will be exactly the same as a target dose amount, some negligible difference is specifically contemplated, and what is considered “negligible” will depend on specific operation conditions.

In one or more embodiments, the delivery mechanism controller 210 may be configured to determine and generate feedback information about delivery actions, such as times of delivery actions and dispensed amounts, without limitation. The feedback information may be generated based on information generated by the delivery mechanism 212 or by the sensors utilized by the delivery mechanism controller 210 to monitor operation of the delivery mechanism 212 (sensors not depicted). For example, sensors to monitor mechanical movement, current consumption, a voltage profile of an electric motor, without limitation may be used. Such information may be logged and provided to and stored at the controller 208, the handheld electronic computing device 138, or the service 150, without limitation, for e.g., later processing or reading, without limitation. For example, the logged information may be processed to determine patterns that may be utilized to determine whether the delivery system 202 is operating as expected (e.g., in a predictable manner, without limitation), and if a difference between actual and expected operation exceeds a set threshold value, the delivery mechanism controller 210 may be updated (e.g., firmware, parameters, or both, of the delivery mechanism controller 210 may be updated, without limitation) to compensate or correct for the difference. Additionally or alternatively to update the firmware or parameters, in a multi-part system, one or more parts including the delivery mechanism controller 210 or the delivery mechanism controller 210 may be indicated as needing replacement (e.g., an alarm or alert is generated at the delivery system 202, the automated medicament delivery system 200, the mobile device or computer in communication therewith, without limitation).

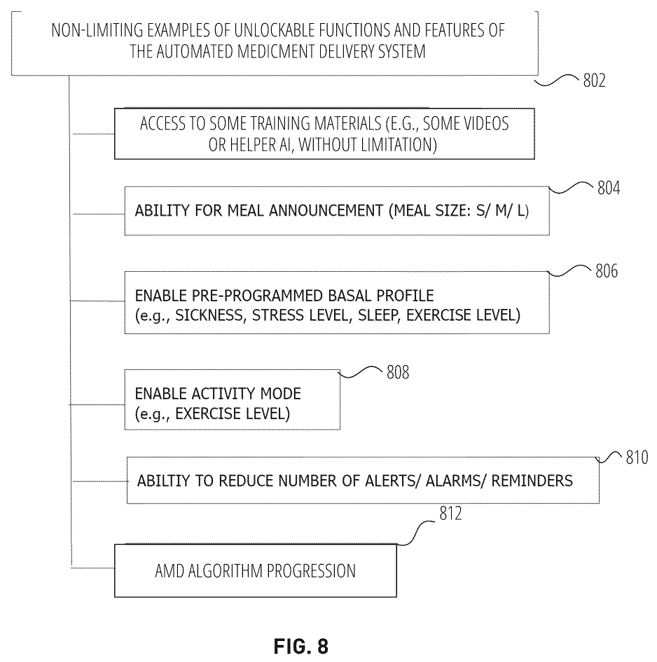

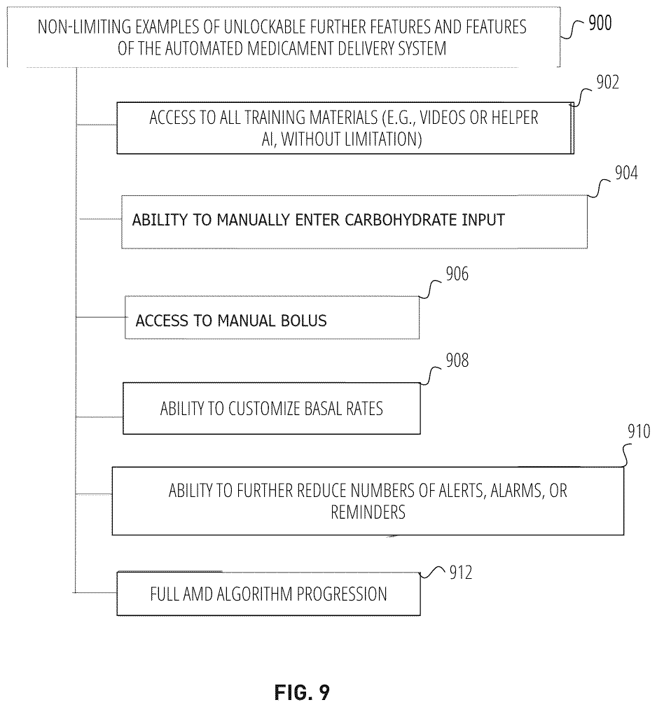

As discussed above, adaptive control logic 222 is capable of changing aspects of delivery modes in response to predetermined conditions. Additionally or alternatively, in one or more embodiments, adaptive control logic 222 may be capable of switching between different delivery mode in response to predetermined conditions. Changing aspects of delivery modes or switching between different delivery modes may unlock capabilities of the system (e.g., activate or enable features or functions of the system, or allow the option to activate or enable features or functions, without limitation) that were previously inaccessible or unavailable to a user or automated medicament delivery system 200, more generally. Non-limiting examples of features or functions that may be unlocked include advanced training content, a more robust UX, a reduced alert burden, bolus calculator and bolus capability, advanced charts with predicted values, and additional features or functions as discussed herein with respect to FIG. 8 and FIG. 9, without limitation.

In one or more embodiments, an automated medicament delivery system that includes examples of adaptive control logic 222 discussed herein may implement a progressive unlock of aspects of a delivery mode in response to predetermined conditions. For example, adaptive control logic 222 may change a UX to allow a user to update parameters (e.g., update values of parameters, without limitation) set at initial configuration or to allow a user to set parameters that were not available at initial configuration (e.g., set unlocked parameters, without limitation). Further, adaptive control logic 222 may allow the automated basal mode to utilize updated parameters and/or unlocked parameters.

As aspects of a delivery mode are unlocked, a user may optionally exert greater influence on the administration of medicament via automated medicament delivery system 200 or system 100 more generally. Additionally or alternatively, various delivery modes or aspects of delivery modes may be allowed as a user evidences greater comfort with automation of medicament administration or treatment of a therapy.

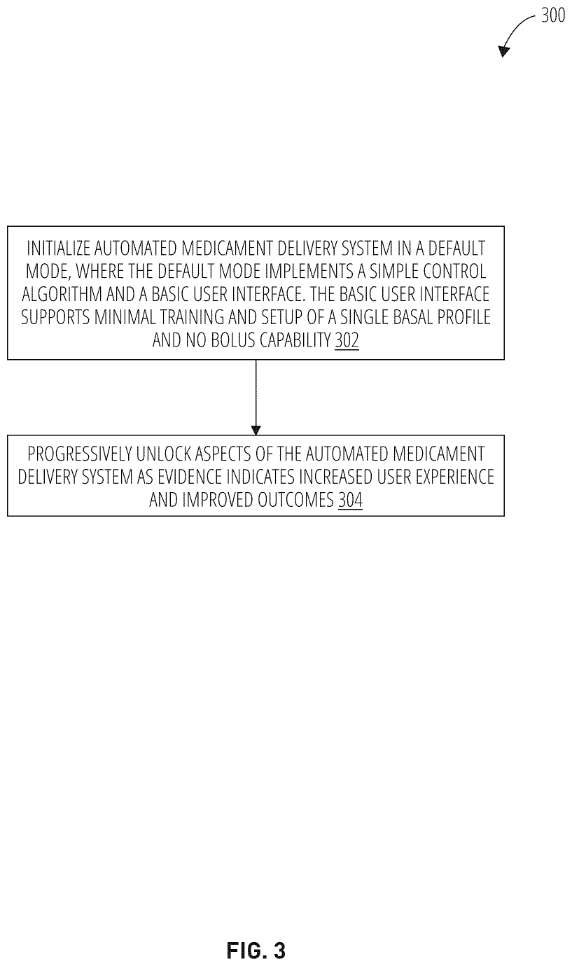

FIG. 3 is a flow diagram depicting a process 300 progressively unlocking features and functions of an automated medicament delivery system as conditions are met, in accordance with one or more embodiments. Although the example process 300 depicts a particular sequence of operations, the sequence may be altered without departing from the scope of this disclosure. For example, some of the operations depicted may be performed in parallel or in a different sequence that does not materially affect the function of the process 300. In other examples, different components of an example device or system that implements the process 300 may perform functions at substantially the same time or in a specific sequence.

According to some examples, the method includes initializing automated medicament delivery system in a default mode, where the default mode implements a simple control algorithm and a basic UX. at operation 302. The basic UX supports minimal training and setup of a single basal profile and does not support a bolus capability. In one or more embodiments, the basic UX supports interaction (e.g., input of information, display of information, without limitation) required to ensure safe operation of the automated medicament delivery system. The basic UX supports input of minimal information, minimal training, minimal display of information about delivery, and no bolus capability. The basic UX may support input of the minimum information about demographics and dosage parameters required to set up a single basal delivery profile for a user, such as age, weight, diabetes type, I:C ratio, or correction factor, without limitation. In some embodiments, the basic UX may support input of a basal rate in addition to, or alternatively to, the I:C ratio and correction factor. By way of non-limiting example of limited support by the basic UX, the basic UX may only present input elements of a GUI for receiving the minimal information.

According to some examples, the method includes progressively unlocking aspects for the automated medicament delivery system as evidence indicates increased user experience and improved outcomes, at operation 304.

In the case of insulin therapy to manage diabetes, evidence of user experience utilized by process 300 may include, as non-limiting examples, duration of time the system has been operating meeting various predetermined thresholds. Evidence of improved outcomes may include, as non-limiting examples, time of A1C within various predetermined ranges (time-in-range (TIR)). For example, A1C≥13: Simple mode with minimal training, no bolus capability, single basal profile, simplified AID algorithm; A1C 12: Video training, meal announcements, more basal profiles, activity mode, advanced AID algorithm; and A1C 10: More video training, carb entry, manual bolus, custom basal rates, full AID algorithm complexity.

Non-limiting examples of features or functions that may be unlocked include meal announcements, bolus calculator, additional basal profiles, activity mode, and AID algorithm adjustments (e.g., personalization of dosage parameters or the AID algorithm more generally, without limitation).

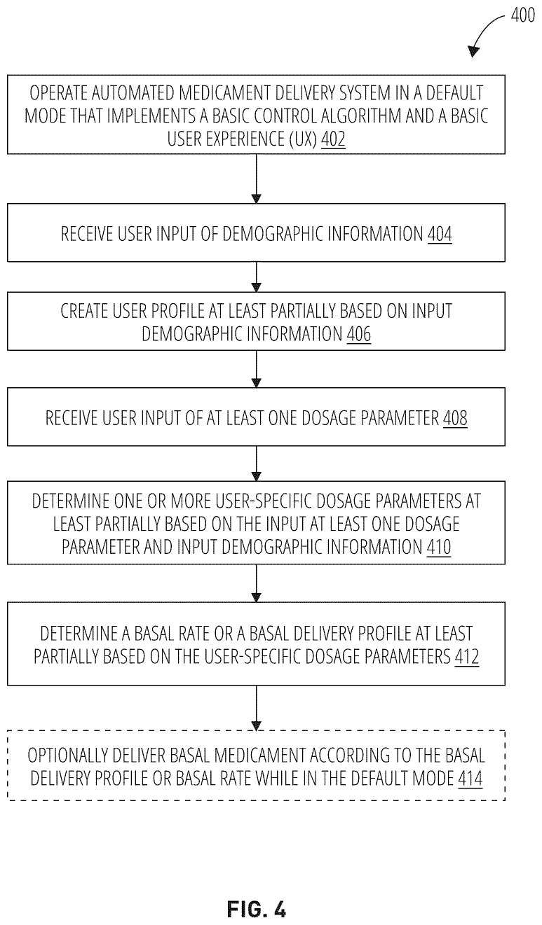

FIG. 4 is a flow diagram illustrating a process 400 to initially configure an automated medicament delivery system, in accordance with one or more embodiments. Although the example process 400 depicts a particular sequence of operations, the sequence may be altered without departing from the scope of this disclosure. For example, some of the operations depicted may be performed in parallel or in a different sequence that does not materially affect the function of the process 400. In other examples, different components of an example device or system that implements the process 400 may perform functions at substantially the same time or in a specific sequence.

During an initial configuration, an automated medicament delivery system in a default mode is configured for automated basal medicament delivery.

According to one or more embodiments, process 400 may include operating an automated medicament delivery system in a default mode that implements a basic control algorithm and a basic user experience (UX), at operation 402. In one or more embodiments, a UI associated with the basic UX supports interaction (e.g., limited to input of information, display of information, without limitation) required to ensure safe operation of the automated medicament delivery system (e.g., required to ensure safe delivery of the medicament, without limitation). In one or more embodiments, the basic UX supports input of minimal amount of information, minimal training, minimal display of information about delivery, and no bolus capability. In one or more examples, the basic UX may support input of the minimum information about demographics (e.g., demographics of the user of the system, without limitation) and dosage parameters required to set up a single basal delivery profile for a user, such as age, weight, or gender without limitation. In the case of an automated insulin delivery system, information about demographics may additionally or alternatively include information about diabetes type, insulin-to-carb (“I:C”) ratio, or correction factor, without limitation. In one or more embodiments, the basic UX may support input of a basal rate in addition to, or alternatively to, the I:C ratio and correction factor.

According to one or more embodiments, process 400 may include receiving user input of demographic information, at operation 404. As discussed above, the demographic information is the minimum information about demographics required to set up a single basal delivery profile for a user, such as age, weight, diabetes type.

According to one or more embodiments, process 400 may include creating a user profile at least partially based on input demographic information, at operation 406.

According to one or more embodiments, process 400 may include receiving user input of at least one dosage parameter at operation 408. The basic UX supports input of a predetermined, limited number of dosage parameters. The basic UX may be configured to prompt a user for, and receive, input of information solely about the at least one dosage parameter. In one or more embodiments, user input of the at least one dosage parameter may be received from, as non-limiting examples: a person with diabetes, a caregiver, a healthcare professional, a remote service (e.g., running on the Cloud or a server, without limitation). As non-limiting examples, the at least one dosage parameter may be a total daily dose (TDD), an average total daily dose (ATDD), a total daily basal dose (TDB), or an average total daily basal dose (ATDB). Alternatively or additionally, in the case of insulin, input user-specific-dosage parameter may include: an insulin-to-carbohydrate (I:C) ratio, a correction factor, or any combination, subcombination, or derivative thereof, without limitation.

According to one or more embodiments, process 400 may include determining one or more user-specific dosage parameters at least partially based on the input of at least one dosage parameter and input demographic information, at operation 410. By way of non-limiting examples, process 400 may determine one or more of: total daily medicament requirement (TDR), total daily basal dose (TDB), average total daily dose of medicament (ATDD), an average basal rate (ABR), or analyte targets. Optionally, process 400 may also determine values for analyte targets, determine values for alert settings at least partially based on the determined analyte targets, and set the alerts.

Alternatively, or additionally, in the case of insulin, determined user-specific-dosage parameters may include: an insulin-to-carbohydrate (I:C) ratio, a correction factor, or any combination, sub-combination, or derivative thereof, without limitation.

According to one or more embodiments, process 400 may include determining a basal rate or a basal delivery profile at least partially based on the determined user-specific dosage parameters, at operation 412. In some cases, TDB or TDD may be utilized to determine the initial baseline basal rate (BBR), an initial carbohydrate-to-insulin ratio (CR), and an initial insulin sensitivity factor (ISF) of a basal delivery profile based on mathematical relationships among and between for BBR, CR, ISF, TDB, and TDD. In some cases, a fixed basal rate may be determined at least partially based on the determined user-specific dosage parameters.

According to one or more embodiments, process 400 may optionally include delivering basal medicament according to the basal delivery profile or basal rate while in the default mode, in optional operation 414.

The automated medicament delivery system automatically administers basal medicament without requiring user supervision or input, and optionally without analyte values. In one or more examples, once automated basal delivery is initiated, parameters, even the limited set of parameters, may not be changed or at least may not be changed via the UX of the user to reduce and/or limit cognitive burden (optionally the parameters could be changed via a UX of a healthcare provider).

In one or more embodiments, upon initiating an automated medicament delivery system according to process 400, other parameters (e.g., parameters other than the limited set of parameters set by the user or based on user input, without limitation) may be previously set (e.g., preset, without limitation) to respective default values. In one or more examples, the default value may be associated with ensuring safe delivery of the medicament. In one or more examples, the “other parameter” are parameters predetermined to not to have significant impact (or stated another way, predetermined to have negligible impact) on the safe delivery of medicament.

Non-limiting examples of other parameters that may be preset to a default value (e.g., a default value associated with ensuring safe delivery of the medicament, without limitation) are: a basal hourly segment value being set at one segment throughout the day, an I:C ratio hourly segment value being set at one segment throughout the day, a correction factor hourly segment value being set at one segment throughout the day, a temporary basal level being set to disabled (not adjustable manually), an extended bolus value being set to disabled (not adjustable manually), a duration of insulin action being set at four hours, a glucose target level being set at 120 mg/dL, an alert/alarm/reminder setting being set to automatic default minimum baseline that is shown to be safe for the user, a blood sugar goal being set to a range of from 70 mg/dL to 180 mg/dL, an “In an AID-enabled system: Mode switching” being set to automated mode only, an “In an AID-enabled system: Exercise mode” being set to disabled (not adjustable manually), or any combination thereof.

Once the automated medicament delivery system has been initialized (set to utilize the initial configuration), the person with diabetes may begin use of the automated medicament delivery system. Over a period of time, the adaptive control logic may change aspects of the delivery mode and/or UX in response to occurrences of predetermined conditions, as discussed below.

As mentioned above, the automated medicament delivery system may include adaptive control logic to progressively enable features based on evidence of user experience and improved outcomes. So, an automated medicament delivery system initialized according to process 300 with limited features or functions may nevertheless have features or functions unlocked.

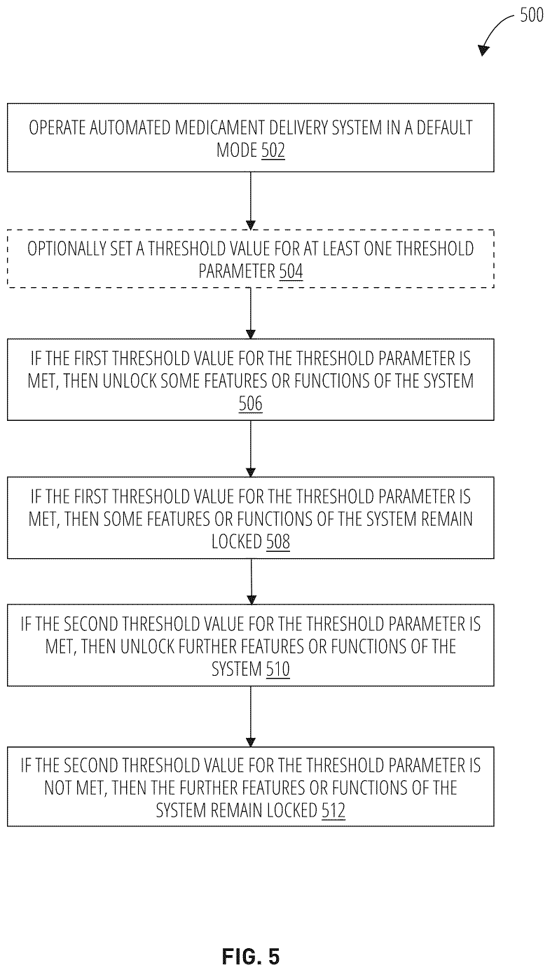

FIG. 5 is a flow diagram depicting a process 500 for progressive unlock of various aspects of automated medicament delivery, in accordance with one or more embodiments. Although the example process 500 depicts a particular sequence of operations, the sequence may be altered without departing from the scope of this disclosure. For example, some of the operations depicted may be performed in parallel or in a different sequence that does not materially affect the function of the process 500. In other examples, different components of an example device or system that implements the process 500 may perform functions at substantially the same time or in a specific sequence.

According to one or more embodiments, process 500 may include operating automated medicament delivery system in a default mode at operation 502.

According to one or more embodiments, process 500 may optionally include setting a threshold value for (e.g., associated with, without limitation) at least one threshold parameter of the system at operation 504. In one or more embodiments, the threshold parameter may be a selected threshold parameter. In one or more embodiments, the at least one threshold parameter may be selected during or after initial set up of the automated medicament delivery system for the user. Additionally or alternatively, the at least one threshold parameter and/or threshold value may be associated with the default mode and selected or set based on the default mode. The at least one threshold parameter and threshold value implement a condition for changing an aspect of the delivery mode or UX. In one or more examples, threshold parameters and threshold values may be pre-set at the adaptive control logic before an automated medicament delivery system is set up or received by a user.

According to one or more embodiments, process 500 may include, if the first threshold value for the selected threshold parameter is met, enabling the option to unlock some features or functions, at operation 506. In one or more embodiments, the adaptive control logic may detect that the threshold value for the selected threshold parameter (“the condition”) is met. After such condition(s) is met, the adaptive control logic may change an aspect of the delivery mode or UX at least partially responsive to observing the condition is met.

In one or more embodiments, an adaptive control logic may organize aspects of a delivery mode or UX that are unlockable according levels that are initiated at the controller by the adaptive control logic in response to various conditions. As non-limiting examples, in a beginning level, the adaptive control logic may allow the user to manually adjust and/or control a few features of the delivery mode and/or UX; while in the higher level, the device may allow the user an ability to manually adjust and/or control more features of the delivery mode and/or UX than the beginning level.

Non-limiting examples of threshold parameters may include, A1C value, a duration of using the device (e.g., cumulative, aggregate, or both), a threshold parameter and value customized by a user, a threshold parameter customized by a healthcare provider, a basal hourly segment, an I:C ratio hour segment, a correction factor hourly segment, temporary basal, extended bolus, duration of insulin action, glucose target level, or any combination or sub-combination thereof. In one or more embodiments, an adaptive control logic may include logic for receiving values indicative of the threshold parameters (e.g., from a healthcare provider, a user, a sensor (e.g., a CGM, a BGM), an internal counter, a delivery system (e.g., delivery system 202, without limitation), without limitation), comparing the received values to the threshold values, and determining whether or not a conditions occurred in response to such comparisons.

According to one or more embodiments, process 500 may include, if the first threshold value of the threshold parameter is met, and some features or functions remain locked at operation 508.

According to one or more embodiments, process 500 may, if the second threshold value for the selected threshold parameter is met, unlock further features or functions at operation 510. In one or more embodiments, the adaptive control logic may detect whether or not the second threshold value for the selected threshold parameter (“the further condition”) is met. The adaptive control logic may unlock further aspects (e.g., further features or functions, without limitation) at least partially responsive to observing the further condition is met.

According to one or more embodiments, process 500 may include, if the second threshold value for the selected threshold parameter is not met, then further features or functions may remain locked, at operation 512. In one or more examples, the adaptive control logic, at least partially responsive to detecting that that second threshold value for the selected threshold parameter is not met, does not unlock further aspects.

While not depicted by FIG. 5, process 500 may continue to monitor and evaluate conditions for unlocking yet further features and functions, the number and types of conditions, features and functions will depend on specific operating conditions/environment.

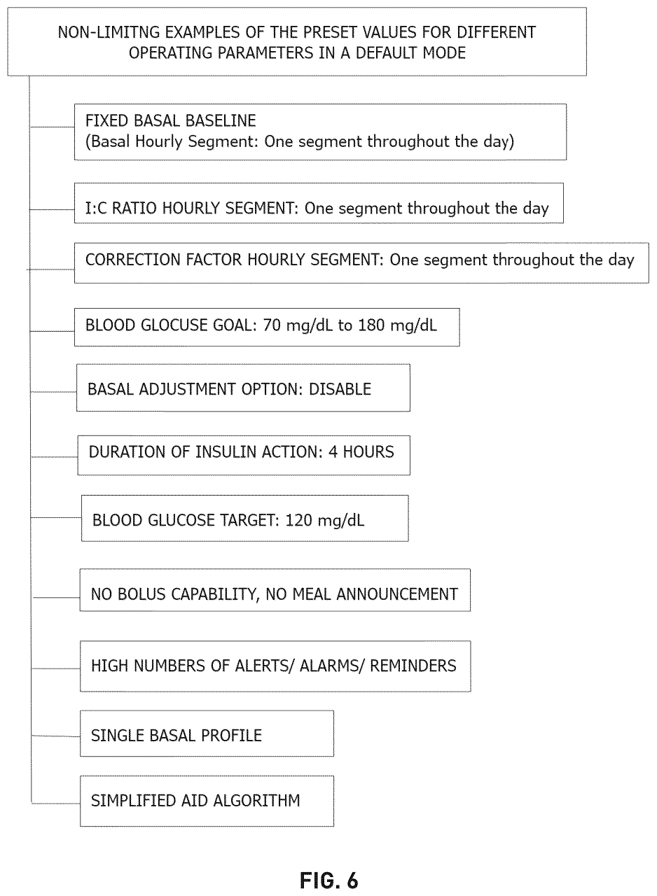

FIG. 6 shows non-limiting examples of values for which numerous operating parameters of for administration of medicament may be preset when the automated medicament delivery device (e.g., AID device equipped with insulin pump, without limitation) operates in a default mode. In various embodiments, one or more operating parameters shown in FIG. 6 are preset to certain values that ensure safe delivery of medicament with minimum or no input from the user of the device.

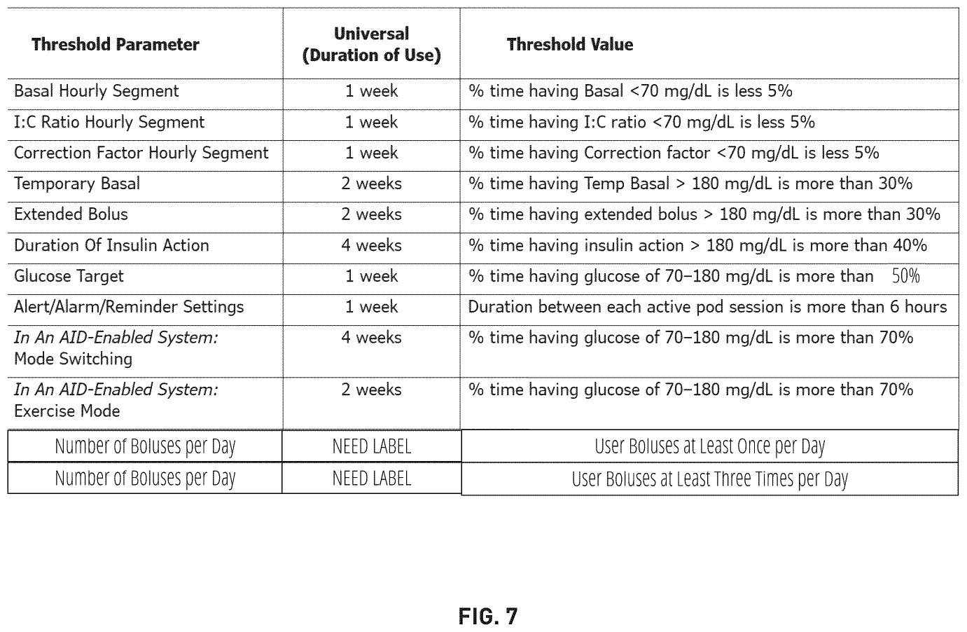

FIG. 7 is a table of non-limiting examples of threshold parameters that may be utilized to adapt a delivery mode and/or UX, according to one or more embodiments. In various embodiments, preset values are selected for one or more operating parameters shown in FIG. 6. Based on the duration of use and/or the values of the threshold parameters per feature category, as detailed in FIG. 7, the individual feature category may be displayed to the user. In some embodiments, such threshold parameters may be calculated continuously from the initiation of the system use and compared against the stated value of the threshold. The feature may be enabled in response to the stated value of the threshold is met beyond the duration of use. These assessments may be repeated periodically, and the feature may be hidden and a recommendation provided to the user to not use the feature if the measurement of the threshold parameter is reduced below the threshold value after initially being above the threshold value. The duration of use and threshold parameters are shown as examples only, and other duration of use and threshold parameters may be utilized additionally or alternatively without exceeding the scope. Further, duration of use and threshold parameters may be modified, further added, and/or removed as needed given specific operating conditions. Further, certain features may be unlocked after only a fixed duration of use without a threshold parameter requirement, and/or after only a threshold parameter requirement is met (after a minimum duration of use) without a duration requirement.

Threshold parameters may include parameters about control, behaviors (e.g., desirable or undesirable behaviors, without limitation), or other life patterns. The specific, non-limiting examples of threshold parameters in FIG. 7 are generally parameters about control. Non-limiting examples of threshold parameters that may be utilized additionally or alternatively to those depicted in FIG. 7 include: frequency of the user-initiated boluses, the frequency of users replacing their pump sites, the frequency of any system alerts that do not get noticed by the user, and other behavioral patterns. Additionally or alternatively to control-based threshold parameters, life pattern-based threshold parameters may be utilized, such as the frequency with which the user may check their history settings, the frequency with which the user encounters a change in time zones, without limitation. Life-based threshold parameters may be utilized to determine whether the user may benefit from additional system features that are otherwise hidden to the user.

In one or more embodiments, behavioral threshold parameters may be evaluated prior to, or in combination with, blood glucose-based threshold parameters for purposes of unlocking features or functions. As non-limiting examples, behavioral threshold parameters may include a duration of pump use exceeding a predetermined period (e.g., one day, without limitation), and a frequency of user-initiated boluses exceeding a predetermined frequency (e.g., at least one bolus per day for a period of one week, or at least three boluses per day for a period of one week, without limitation). Such behavioral threshold parameters may be utilized to initially unlock some features (e.g., features shown in FIG. 8, without limitation), and blood glucose-based threshold parameters may be utilized to unlock additional features (e.g., features shown in FIG. 9, without limitation) thereafter.

FIG. 8 provides non-limiting examples of unlockable features or functions of an automated medicament delivery system, according to one or more examples. The exemplary features include: access to some training materials (e.g., some videos or helper AI, without limitation) (802), ability for meal announcement (e.g., meal size: S/M/L, without limitation) (804), enable pre-programmed basal profile (e.g., for sickness, stress level, sleep, exercise level, without limitation) (806), enable activity mode (e.g., exercise level, without limitation) (808), ability to reduce the number of alerts, alarms, and/or reminders (810), and automated medicament delivery (AMD) algorithm progression (812). One or more of the features may be available for the delivery mode and/or the UX.

In various embodiments, one or more features or functions shown in FIG. 8 are available for the user to adjust and/or control by user interface (e.g., via an input device 130 of the automated medicament delivery system 114 of the system 100 shown in FIG. 1, without limitation) upon unlock as discussed herein.

A helper artificial intelligence (AI), or “helper AI,” is an intelligent virtual assistant that provides interactive, real-time support to users training on an automated medicament delivery system. In one or more embodiments, the helper AI is trained on (here, the “trained on” means the helper AI's underlying machine learning models are developed and fine-tuned using) a curated dataset comprising instructional materials such as user manuals, training videos, interactive websites, troubleshooting guides, quizzes, and system operation documentation. A helper AI is designed to enhance user understanding, reduce the learning curve, and ensure safe and effective usage of the system. Capabilities of a helper AI may include, but are not limited to, one or more of: natural language interface, contextual awareness, multimedia support, error detection and resolution guidance, interactive training modules, searchable knowledge base, personalized learning path. Components of the helper AI may include, but are not limited to, one or more of: machine learning core, training material integration, and interactive interface.

Capabilities powered by the machine learning core may include, but are not limited to, one or more of: natural language processing, computer vision, contextual and conditional awareness, inference capabilities for error detection and resolution guidance, and recommendation algorithms. Training material integration may include, but is not limited to, one or more of: ingest and index of instructional material (e.g., text, video, image, or audio, without limitation).

Capabilities of the interactive interface may include, but are not limited to: accessibility via multiple platforms (e.g., mobile app, tablet, wearable device, or on-delivery device screen, without limitation), or text-based chat, voice command support, verbal and text based instructions, guided monitoring or other interactive feedback monitoring (passive or active training, context-aware feedback, proactive observation, real-time performance coaching, assistive monitoring), optional augmented reality and/or virtual reality overlays.

FIG. 9 provides non-limiting examples 900 of features or functions of an automated medicament delivery system available for unlocking, according to one or more examples. The exemplary features or functions include: access to all training materials (902), ability to manually enter carbohydrates (meal announcement) (904), access to manual bolus (906), ability to customize basal rates (908), ability to further change alarms and alerts and/or reminders (e.g., set the number of alarms and alerts and/or reminders, set conditions and types of alarms and alerts and/or reminders, without limitation) (910), and full automated medicament delivery (AMD) algorithm progression (912). While not depicted by FIG. 9 additional non-limiting examples of features or functions of the automated medicament delivery system available for further unlock may include: ability to adjust glucose set-point, ability to enable extended boluses where initially a portion of a bolus is delivered followed by subsequent delivery of the remaining portion of the bolus after a period of time or over a period of time, ability to enable automatic correction bolus delivery, ability to enable automatic meal detection and meal bolus delivery, ability to adjust minimum and/or maximum size of a correction bolus, or ability to adjust minimum and/or maximum size of a meal bolus.

In one or more embodiments, the features shown in FIG. 9 may be incrementally made available to the user as conditions are detected.

In one or more embodiments, one or more features or functions shown in FIG. 8 are available for the user to adjust and/or control by user interface (e.g., via an input device 130 of the automated medicament delivery system 114 of the system 100 shown in FIG. 1, without limitation) upon unlock as discussed herein.

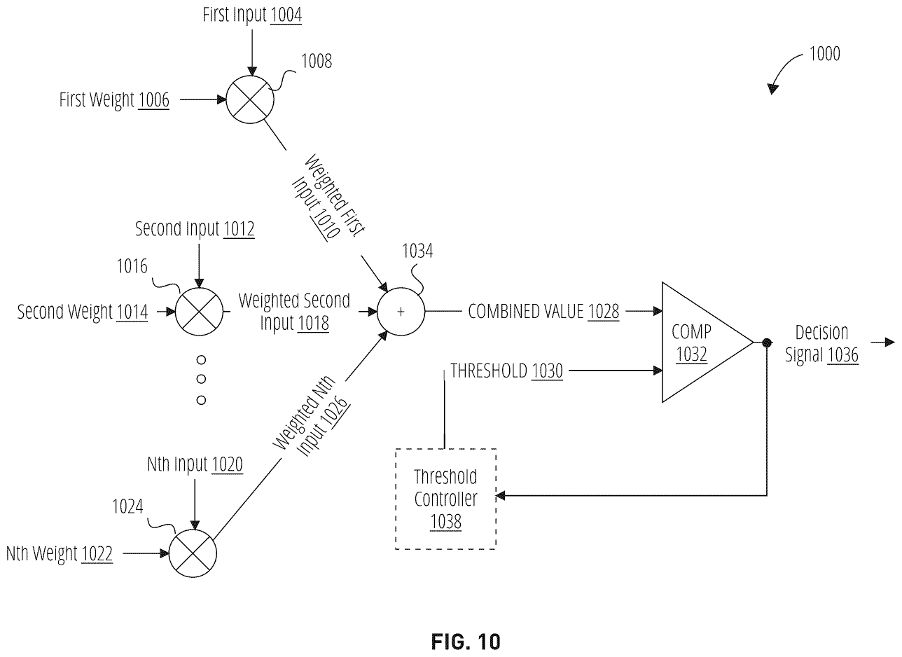

FIG. 10 is a flow diagram of a condition processing chain 1000, in accordance with one or more embodiments. Condition processing chain 1000 evaluates multiple threshold parameters (e.g., threshold parameters identified in FIG. 7, without limitation), individually or together, to determine whether evidence indicates increased user experience and improved outcomes with an automated medicament delivery system sufficient to progressively unlock aspects of the automated medicament delivery system (e.g., aspects identified in FIG. 8 or FIG. 9, without limitation).

In one or more embodiments, various inputs (first input 1004, second input 1012, and Nth input 1020) are summed (via summer 1034) or weighted (e.g., weighted by multiplier 1008, multiplier 1016, or multiplier 1024 with first weight 1006, second weight 1014, or Nth weight 1022 to produce weighted first input 1010, weighted second input 1018, or weighted nth input 1026, respectively) and summed (via summer 1034). The combined value 1028 are compared (via COMP 1032) to threshold 1030, and condition processing chain 1000 outputs a decision signal 1036 at least partially based on the comparison. If the combined value 1028 exceeds threshold 1030 (meets the condition) then condition processing chain 1000 outputs decision signal 1036 with a first value (first value represents “TRUE”), and if the combined value 1028 does not exceed threshold 1030 (does not meet the condition) then condition processing chain 1000 outputs decision signal 1036 with a second value that is different than the first value (second value represents “FALSE”).

In some cases, values of inputs (first input 1004, second input 1012, and Nth input 1020) to condition processing chain 1000 may reflect binary determinations e.g., positive, TRUE, or ‘1’; or negative, FALSE, or ‘0’. In other cases, values of inputs (first input 1004, second input 1012, and Nth input 1020) to condition processing chain 1000 may reflect a score or degree to which a specific parameter is met. As a non-limiting example of the binary determination case, if the % time having Basal<70 mg/dL is less than 5% a score of 1 is assigned, and if the % time having Basal<70 mg/dL is greater than 5% a score of 0 is assigned. As a non-limiting example of the scoring case, if the % time having Basal<70 mg/dL is greater than 5% but less than 7.5%, a score of 0.5 is assigned (a score of 0 would have been assigned in the binary determination case), and if the % time having Basal<70 mg/dL less than 5% a score of 1 is assigned. Thus, some credit may be given to reflect partially meeting a threshold parameter.

The values of weights applied by condition processing chain 1000 (e.g., first weight 1006, second weight 1014, and Nth weight 1022) and the threshold 1030 may be preset. In some cases, it may be desirable that each input be fully true for condition processing chain 1000 to generate a positive (TRUE) decision signal 1036. In such a case, each weight may be preset equal to 1 and the threshold 1030 preset equal to the sum of the weights. In this specific example, setting the weights and threshold appropriately ensures that the system only generates a positive decision signal 1036 when all inputs are respectively fully true.

In some cases it may be desirable that at least a specific number of the inputs be fully true for condition processing chain 1000 to generate a positive decision signal 1036. In such a case, each weight may be preset equal to 1 and the threshold 1030 preset equal to the sum of the threshold number of weights that must be true for condition processing chain 1000 to generate a positive decision signal 1036. In this specific example, setting the weights and threshold appropriately ensures that the system only generates a positive decision signal 1036 when the threshold number of inputs are respectively fully true.

In some cases, unlock of specific aspects may be triggered by specific threshold parameters being met, and additionally or alternatively, unlock of specific aspects may be triggered by the combined value exceeding specific, different thresholds. In the latter case (unlock of specific aspects may be triggered by combined values exceeding specific, different thresholds), the same or different sets of threshold parameters may combine and compared to determine whether or not specific aspects should be unlocked.

As a non-limiting example, in some cases, threshold 1030 may be adjustable allowing the value of threshold 1030 to change dynamically. In one or more embodiments, condition processing chain 1000 may include optional threshold controller 1038 to adjust threshold 1030 (e.g., adjust a value of threshold 1030, without limitation) in response to a positive decision signal 1036 (e.g., in response to a transition from a negative or neutral decision signal 1036 to a positive decision signal 1036, without limitation). Specifically, threshold controller 1038 may increase the value of threshold 1030 in response to a positive decision signal 1036 to ensure that a subsequent values of decision signal 1036 are based on a comparison of combined value 1028 against a higher threshold 1030. This approach requires a higher combined value 1028 to satisfy some conditions to unlock aspects of an automated medicament delivery system, reflecting continuous improvement-e.g., through increased user engagement and improved outcomes.

In one or more examples, adaptive control logic 222 may include one or more condition processing chains 1000, respectively to evaluate a user's progress meeting conditions for unlocking aspects of the automated medicament delivery system. As non-limiting examples, adaptive control logic 222 may include an instance of condition processing chain 1000 for each aspect capable of being unlocked, and additionally or alternatively, may include a threshold controller 1038 to adjust values of thresholds 1030 to accommodate different conditions for different aspects.

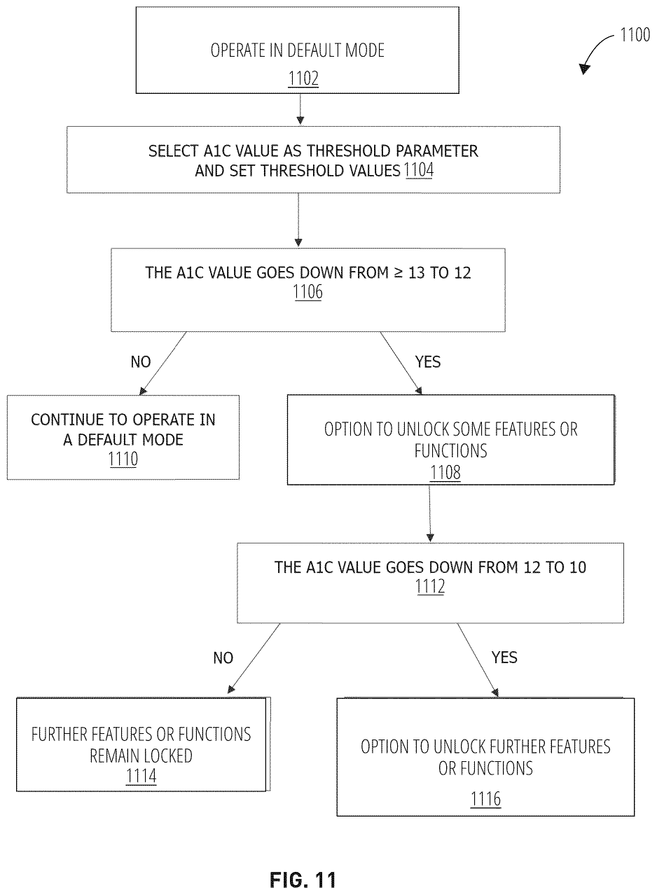

FIG. 11 is a flow diagram of a process 1100 for automated delivery of medicament in accordance with one or more embodiments. FIG. 11 depicts a specific use case where the A1C value is used as the threshold parameter.

In one or more embodiments, process 1100 may include, at operation 1102, the user utilizing the automated medicament delivery device (e.g., AID device, without limitation) in a default mode.

In one or more embodiments, process 1100 may include, at operation 1104, the A1C value is selected as a threshold parameter and certain threshold values of the A1C values are set as the condition for which an adaptive control logic of the device may adapt a delivery mode and/or user interface at least partially responsive to observing the condition is met.

As a non-limiting example, the user may have an A1C value of more than 13 when first used the device, which corresponds to severely elevated case of diabetes with high risk of complications, such as heart attack, stroke, blindness, kidney failure, amputation, without limitation. In one or more embodiments, process 1100 may include, at operation 1106, the first threshold value may be set at the A1C value of 12.

When the user properly practices the insulin treatment regimen, the A1C value of the user may decrease from more than 13 to about 12. In one or more embodiments, process 1100 may include, at operation 1108, an adaptive control logic of the device may adapt a delivery mode and/or UX (e.g., unlocks, without limitation) at least partially responsive to observing the condition is met. The automated medicament delivery device (e.g., AID device, without limitation) may provide an option for the user to unlock some features of the delivery mode and/or user interface.

In one or more embodiments, process 1100 may include, at operation 1110, if the A1C value of the user does not go down to 12, then the adaptive control logic of the device does not adapt delivery mode and/or user interface. Here, features of the delivery mode and/or user interface will remain locked and the user has no choice but to continue with the default mode of delivery and/or user interface.

In one or more embodiments, the method of automated delivery of medicament includes triggering an alert function of the automated medicament delivery device once the threshold parameter is met, to inform the user of an option for the delivery mode and/or user interface.

In one or more embodiments, process 1100 may include, at operation 1112, as the user properly continues with an insulin treatment regimen, the A1C value of the user may further go down from about the value of 12 to about 10 as shown.

In one or more embodiments, process 1100 may include, at operation 1116, when the second threshold value may be met, the automated medicament delivery device (e.g., AID device, without limitation) may provide an option for the user to unlock further features of the delivery mode and/or user interface.

In one or more embodiments, process 1100 may include, at operation 1114, if the A1C value of the user is still in a range of from 12 to more than 10, then the further features of the delivery mode and/or user interface will remain locked.

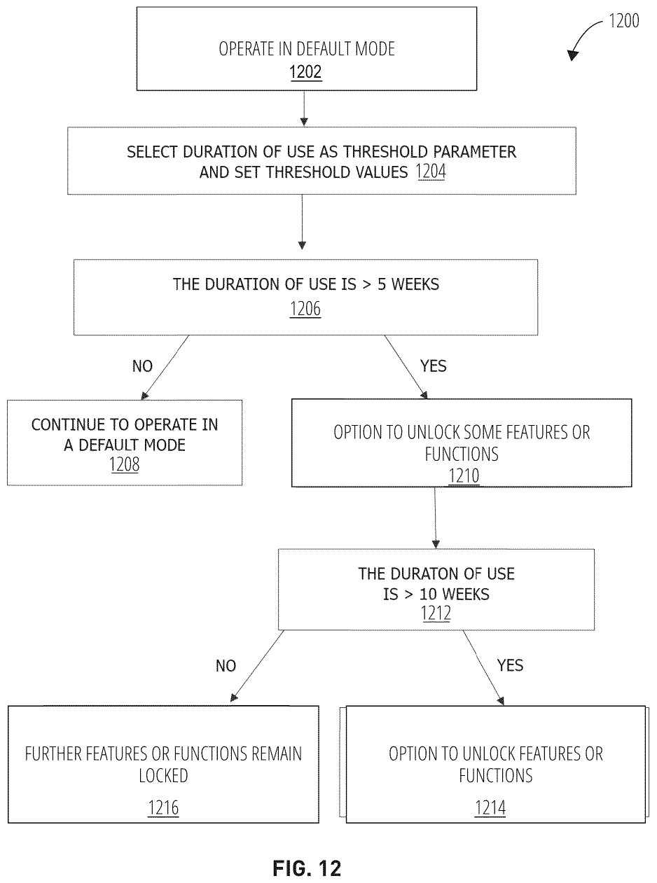

FIG. 12 is a flow diagram of a process 1200 for automated delivery of medicament in accordance with one or more embodiments, wherein the duration of using the automated medicament delivery device is used as the threshold parameter.

In one or more embodiments, process 1200 may include, at operation 1202, the automated medicament delivery device operating in the default mode of delivery and/or user interface.

In one or more embodiments, process 1200 may include, at operation 1204, the duration of using the device is selected as a threshold parameter and certain threshold values of the duration of use are set as conditions for which the adaptive control logic of the device may adapt a delivery mode and/or user interface at least partially responsive to observing the condition is met.

In one or more embodiments, process 1200 may include, at operation 1206, the threshold value of duration of use may be set at more than 5 weeks. Namely, after a continuing use of the automated medicament delivery device for more than 5 weeks, the adaptive control logic of the device may adapt a delivery mode of delivery and/or user interface at least partially responsive to observing the condition is met.

In one or more embodiments, process 1200 may include, at operation 1210, some features of the delivery mode and/or user interface may be unlocked and/or the user has an option to unlock some features or functions of the delivery mode and/or user interface. For example, after a continuing use of more than 5 weeks, the user may be familiar with the device and may not feel overwhelmed by the user interfaces offered by the device.

In one or more embodiments, process 1200 may include, at operation 1208, if the duration of use of the device is less than 5 weeks, then the features of delivery mode and/or user interface will remain locked. The user cannot change the delivery mode and/or user interface from a default mode, e.g., the user interface with the device is kept at minimal or none to ensure safe delivery of medicament to the user, without limitation.

In one or more embodiments, process 1200 may include, at operation 1212, determining if the duration is more than 10 weeks. If the duration is more than 10 weeks then process 1200 may include, at operation 1214, allowing the user the option to unlock further features or functions of the delivery mode and/or user interface as shown. Without limitation, the user may have an ability for meal announcement and to choose the meal size accordingly (S/M/L), or the user may tailor the administration of medicament according to the physical activity level (e.g., lower level of administered insulin during exercise), the wellness level of the user (e.g., higher level of administered insulin during sickness or stress), or even at a certain time period (e.g., lower level of administered insulin during sleep at night). In one or more embodiments, process 1200 may include, at operation 1216, if the duration of use is less than 10 weeks, then the user may not have an ability to unlock any further features of the delivery mode and/or user interface.

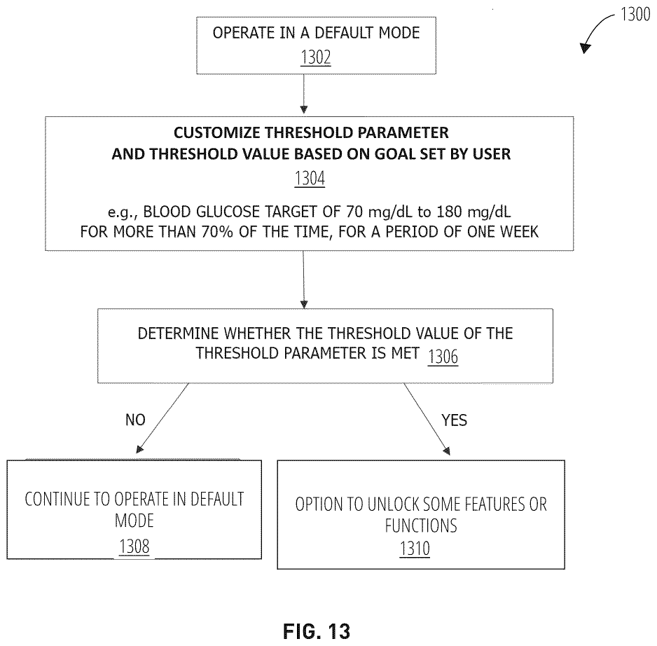

FIG. 13 is a flow diagram of a process 1300 for automated delivery of medicament in accordance with one or more embodiments. In process 1300, a goal set by the user of the automated medicament delivery device is used as the basis for the threshold parameter and threshold value.

In one or more embodiments, process 1300 may include, at operation 1302, operating the automated medicament delivery device (e.g., AID device, without limitation) in a default mode and/or user interface. While operating in the default mode, at least one operating parameter of the device may be set to a preset value to ensure safe delivery of the medicament, as discussed above with respect to FIG. 6.

In one or more embodiments, process 1300 may include, at operation 1304, customizing the threshold parameter and the threshold value based on a goal set by the user. The user may have an ability to set a performance goal for user well-being, and to select both the threshold parameter to be monitored and the threshold value to be achieved. As a non-limiting example, the user may set a goal of maintaining a blood glucose level within a target range of 70 mg/dL to 180 mg/dL for more than 70% of the time, over a continuous period of one week. In such an example, the threshold parameter is the percentage of time that the blood glucose level is within the target range of 70 mg/dL to 180 mg/dL, and the threshold value is 70% over a period of one week. Other threshold parameters and threshold values may be customized by the user based on user-specific goals without exceeding the scope of the disclosure. In one or more embodiments, the user-customized threshold parameter and threshold value may be subject to review and/or approval by a healthcare professional to ensure safe delivery of the medicament.

In one or more embodiments, process 1300 may include, at operation 1306, the adaptive control logic determining whether the threshold value of the threshold parameter customized by the user is met. The adaptive control logic may continuously or periodically evaluate data captured by the automated medicament delivery device (e.g., analyte data from the analyte sensor 102, without limitation) against the user-customized threshold value.

In one or more embodiments, process 1300 may include, at operation 1308, continuing to operate in the default mode and/or user interface if the threshold value for the selected threshold parameter is not met. In such a case, features of the delivery mode and/or user interface remain locked and the user continues with the default mode of delivery and/or user interface until the user-customized threshold value is achieved.

In one or more embodiments, process 1300 may include, at operation 1310, the automated medicament delivery device (e.g., AID device, without limitation) providing an option for the user to unlock some features or functions of the delivery mode and/or user interface, at least partially responsive to the adaptive control logic determining that the threshold value for the user-customized threshold parameter is met. Non-limiting examples of features or functions that may be unlocked include those discussed above with respect to FIG. 8 and FIG. 9. In one or more embodiments, an alert function of the automated medicament delivery device may be triggered to inform the user of the option to unlock features or functions of the delivery mode and/or user interface upon the user-customized threshold value being met.

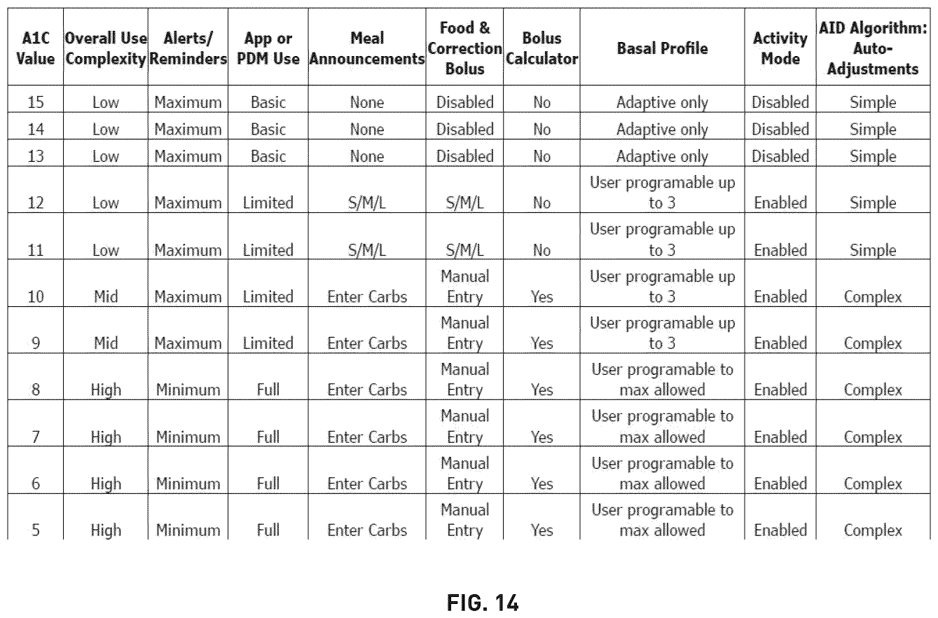

FIG. 14 shows non-limiting examples of the combinations of some features in the delivery mode and/or user interface when the user has achieved different levels of A1C values. FIG. 14 illustrates how feature availability may progressively change across a range of A1C values from 15 down to 5 across multiple feature categories, including overall use complexity, alerts and reminders, application or personal diabetes management (PDM) use, meal announcements, food and correction bolus, bolus calculator, basal profile, activity mode, and AID algorithm auto-adjustments. The specific A1C values, feature categories, and feature configurations shown in FIG. 14 are non-limiting examples, and other A1C values, feature categories, and feature configurations may be utilized without exceeding the scope of the disclosure.

In one or more embodiments, when the user has a relatively high A1C value (e.g., an A1C value of 15, 14, or 13, without limitation), the automated delivery of medicament may be set to a default mode of delivery and/or user interface with a low overall use complexity. At such A1C values, the delivery mode and/or user interface may include: a maximum number of alerts, alarms, and/or reminders to ensure that the user complies with the required criteria for a proper administration of medicament; a basic application or PDM capability; no meal announcement capability; food and correction bolus disabled; no bolus calculator; a single adaptive-only basal profile as defaulted by the device; activity mode disabled; and a simple AID algorithm for auto-adjustments, without limitation.

In one or more embodiments, as the A1C value of the user decreases to a first intermediate range (e.g., an A1C value of about 12 or about 11, without limitation), the adaptive control logic of the device may adapt a delivery mode and/or user interface at least partially responsive to observing the condition is met. At such A1C values, the overall use complexity may remain low, and some features of the delivery mode and/or user interface may be unlocked. As non-limiting examples, features that may be unlocked or changed at this level include: a limited application or PDM capability (as compared to the basic capability at higher A1C values); an ability for meal announcement based on meal size (e.g., S/M/L, without limitation); food and correction bolus based on meal size (e.g., S/M/L, without limitation); an ability for the user to select up to three (3) programmable basal profiles (as compared to adaptive-only at higher A1C values); an activity mode enabled for the user to interface with the device about the level of activity engaged by the user at any particular moment; and a simple AID algorithm for auto-adjustments. At this level, a bolus calculator may not yet be available, and the number of alerts, alarms, and/or reminders may remain at a maximum setting.

In one or more embodiments, as the A1C value of the user further decreases to a second intermediate range (e.g., an A1C value of about 10 or about 9, without limitation), additional features of the delivery mode and/or user interface may be unlocked. At such A1C values, the overall use complexity may increase to a mid level. As non-limiting examples, features that may be unlocked or changed at this level include: a limited application or PDM capability; an ability to enter meal announcements by entering carbohydrate values (as compared to meal size only at higher A1C values); food and correction bolus by manual entry (as compared to meal size-based at higher A1C values); a bolus calculator enabled; an ability for the user to select up to three (3) programmable basal profiles; activity mode enabled; and a complex AID algorithm for auto-adjustments (as compared to the simple algorithm at higher A1C values). At this level, the number of alerts, alarms, and/or reminders may transition from a maximum setting to a maximum or minimum setting depending on the specific A1C value achieved.

In one or more embodiments, as the A1C value of the user further decreases to a lower range (e.g., an A1C value of about 8, about 7, about 6, or about 5, without limitation), yet further features of the delivery mode and/or user interface on the automated medicament delivery device may be unlocked. At such A1C values, the overall use complexity may be high. As non-limiting examples, features that may be available at this level include: a full application or PDM capability; an ability to enter meal announcements by entering carbohydrate values; food and correction bolus by manual entry; a bolus calculator enabled; an ability for the user to select up to a maximum number of programmable basal profiles (as compared to up to three at higher A1C values); activity mode enabled; a complex AID algorithm for auto-adjustments; and a minimum number of alerts, alarms, and/or reminders, without limitation.

As illustrated by FIG. 14, the progressive unlocking of features across decreasing A1C values provides a graduated approach to increasing user control over the automated medicament delivery system. In one or more embodiments, the adaptive control logic may evaluate the A1C value of the user and determine which combination of features, from among the feature categories shown in FIG. 14, are to be made available to the user in the delivery mode and/or user interface. The specific A1C values at which particular features become available may be preset, set by a healthcare professional, customized by the user, or any combination thereof, without limitation.

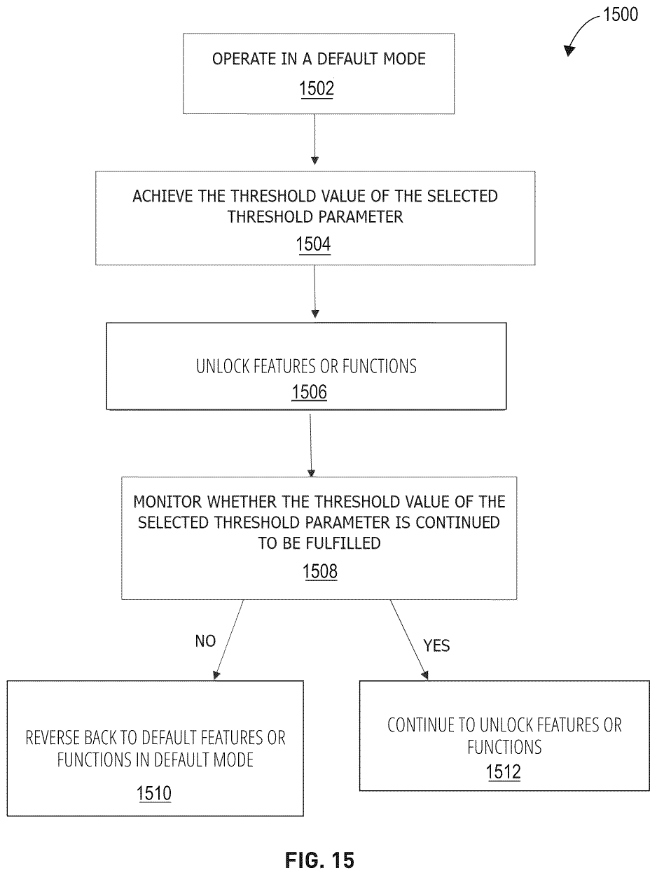

FIG. 15 is a flow diagram of a process 1500 for automated delivery of medicament in accordance with one or more embodiments. Process 1500 illustrates a scenario in which the automated medicament delivery device detects that a threshold value for a selected threshold parameter is no longer being met after features or functions have been unlocked, and reverses operation back to a default mode and/or user interface. Although the example process 1500 depicts a particular sequence of operations, the sequence may be altered without departing from the scope of this disclosure. For example, some of the operations depicted may be performed in parallel or in a different sequence that does not materially affect the function of the process 1500.

In one or more embodiments, process 1500 may include, at operation 1502, operating the automated medicament delivery device (e.g., AID device, without limitation) in a default mode and/or user interface, wherein at least one operating parameter of the device is set to a preset value to ensure safe delivery of the medicament, as discussed above.

In one or more embodiments, process 1500 may include, at operation 1504, achieving the threshold value of the selected threshold parameter. The threshold value may be achieved, for example, when the adaptive control logic determines, based on data captured by the automated medicament delivery device (e.g., analyte data from the analyte sensor 102, without limitation), that the threshold value for the selected threshold parameter has been met, as discussed above with respect to FIGS. 5, 11, 12, and 13.

In one or more embodiments, process 1500 may include, at operation 1506, unlocking features or functions of the delivery mode and/or user interface at least partially responsive to the threshold value being achieved. Non-limiting examples of features or functions that may be unlocked include those discussed above with respect to FIG. 8 and FIG. 9. In one or more embodiments, the user may be presented with an option to unlock one or more features or functions, and may accept or decline the option.

In one or more embodiments, process 1500 may include, at operation 1508, monitoring whether the threshold value of the selected threshold parameter continues to be fulfilled after features or functions have been unlocked. The adaptive control logic may continuously or periodically evaluate data captured by the automated medicament delivery device (e.g., analyte data from the analyte sensor 102, without limitation) against the threshold value to determine whether the user continues to meet the threshold value. For example, the analyte sensor 102 of FIG. 1 may communicate via its communication equipment 112 to the communication equipment 124 of the automated medicament delivery system 114 that the threshold value is or is no longer being met, and the processor 118 of the automated medicament delivery system 114 or the controller 208 of FIG. 2 may determine whether the threshold value continues to be fulfilled.

In one or more embodiments, process 1500 may include, at operation 1512, continuing to unlock features or functions of the delivery mode and/or user interface if, at operation 1508, the adaptive control logic determines that the threshold value of the selected threshold parameter continues to be fulfilled. In such a case, the user may retain access to the previously unlocked features or functions and may be eligible for progressive unlocking of additional features or functions as further conditions are met, as discussed above with respect to FIG. 5.

In one or more embodiments, process 1500 may include, at operation 1510, reversing back to default features or functions in the default mode if, at operation 1508, the adaptive control logic determines that the threshold value of the selected threshold parameter is no longer being fulfilled. In such a case, the previously unlocked features or functions may be locked and the automated medicament delivery device may revert to the default mode and/or user interface to ensure safe delivery of the medicament to the user. In certain embodiments, the user may not resume the unlocked delivery mode and/or user interface until the threshold value is met again. In certain embodiments, the user may not resume the unlocked delivery mode and/or user interface unless and until receiving an approval from one or more healthcare professionals, in addition to fulfilling the threshold value. In one or more embodiments, one or more healthcare professionals may have real-time access to the analyte values of the selected threshold parameter, and therefore may modify the delivery mode and/or user interface of the automated medicament delivery device promptly if any correction is needed to ensure safe medicament delivery to the user.

According to one or more embodiments of the disclosed method of automatic delivery of medication, the user interface and interaction modes of the automated medicament delivery device (e.g., insulin pumps in the AID device, without limitation) are programmed to a default mode that requires minimum user interface with the device. Once the user meets certain threshold value for the selected threshold parameters, the automated medicament delivery device may present the user with an option to unlock some features of delivery mode and/or user interface, which may enable the user to alter the settings, activate new settings, and/or gain more access to the user interface of the device.

In some embodiments, when the features of delivery mode and/or user interface are unlocked and accessible to the user, the increased complexity of each setting may be introduced to the user with a brief tutorial module consisting of a looping video.

The embodiments described above and illustrated in the accompanying drawings do not limit the scope of the disclosure, which is encompassed by the scope of the appended claims and their legal equivalents. Any equivalent embodiments are within the scope of this disclosure. Indeed, various modifications of the disclosure, in addition to those shown and described herein, such as alternate useful combinations of the elements described, will become apparent to those skilled in the art from the description. Such modifications and embodiments also fall within the scope of the appended claims and equivalents.

What is claimed is:

1. A method of automated delivery of medicament, comprising:
operating an automated medicament delivery device or system (automated medicament delivery system) in a default mode or with a basic user experience, wherein at least one operating parameter of the automated medicament delivery system is preset to a value associated with ensuring safe delivery of the medicament; and
unlocking a capability of the system for a user of the system to manually adjust or control at least one operating parameter for administration of the medicament once a threshold value for at least one threshold parameter of the system is met.

2. The method of claim 1, wherein setting the threshold value for the at least one selected threshold parameter comprises setting a threshold value for an A1C value parameter.

3. The method of claim 2, wherein setting the threshold value for the at least one selected threshold parameter comprises setting a threshold value for a duration of use parameter.

4. The method of claim 1, wherein setting the threshold value for the at least one selected threshold parameter is performed by healthcare professionals.

5. The method of claim 1, wherein setting the threshold value for the at least one selected threshold parameter is performed by the user of the system.

6. The method of claim 1, wherein a capability for a user not to proceed with manually adjusting and/or controlling at least one operating parameter of the automated medicament delivery system is also unlocked once the threshold value for the at least one selected threshold parameter is met.

7. The method of claim 1, wherein the automated medicament delivery system automatically reverts back to the default mode and/or user interface upon the threshold value for the at least one selected threshold parameter no longer being met.

8. A method of automated medicament delivery, comprising:
utilizing data captured by an automated medicament delivery device or system (automated medicament delivery system) to determine whether a threshold value for a threshold parameter is met; and
providing a user of the automated medicament delivery system, once the threshold value for the threshold parameter is met, a capability to unlock a delivery mode and/or user experience (UX) of the automated medicament delivery system to allow manual adjustment and/or control over at least one operating parameter for administration of the medicament.

9. The method of claim 8, wherein the medicament comprises long-acting insulin, rapid-acting insulin, glucagon-like peptide-1 receptor agonist (GLP-1), glucose-dependent insulinotropic polypeptide (GIP), insulin substitute, hormone, or any combination thereof.

10. The method of claim 8, wherein the automated medicament delivery system comprises an automated insulin delivery system.

11. The method of claim 8, wherein the threshold parameter comprises an A1C value, a duration of using the system, a glucose level, a threshold parameter customized by the user, a threshold parameter customized by healthcare professionals, or any combination thereof.

12. The method of claim 8, comprising operating the automated medicament delivery system in a default mode and/or user interface when the threshold value for the threshold parameter is not met,
wherein in the default mode and/or user interface, the system automatically adjusts and/or controls delivery of medicament to achieve a selected target parameter with minimal user interface with the system.

13. The method of claim 8, comprising triggering an alert function of the automated medicament delivery system once the threshold value is met.

14. A method of automated delivering of medicament, comprising:
establishing a preset value of a default parameter on an automated medicament delivery system to ensure safe delivery of the medicament, when the system is first initiated; and
allowing a user interface with the system when a threshold value of a selected threshold parameter is met, so that the user has a capability to manually adjust and/or control at least one operating parameter of an administration of the medicament.

15. The method of claim 14, comprising selecting the threshold parameter and setting the threshold value for the threshold parameter.

16. The method of claim 14, wherein the default parameter comprises a basal hourly segment value, an I:C ratio hourly segment value, a correction factor hourly segment value, a temporary basal level, an extended bolus value, a duration of insulin action, a glucose target level, an alert/alarm/reminder setting, an analyte level goal, or any combination or sub-combination thereof.

17. A method of automated delivering of medicament, comprising:
setting a condition for a threshold value of a selected threshold parameter; and
adapting a delivery mode and/or user interface at least partially responsive to observing the condition is met.

18. An apparatus or system, comprising:
at least one processor; and
a memory to store instructions that, upon execution by the at least one processor, configure the apparatus to:
operate an automated medicament delivery device in a default mode and/or user interface, wherein at least one operating parameter of the device is set to a preset value to ensure safe delivery of the medicament;
set a threshold value for at least one selected threshold parameters; and
unlock a capability for the device or a user of the device to adjust or control at least one operating parameter for an administration of the medicament at least partially responsive to the threshold value for the at least one threshold parameter being met.

19. The apparatus of claim 18, wherein the at least one processor is configured to revert the automated medicament delivery system to the default mode upon determining that the threshold value for the at least one threshold parameter is no longer met.

20. The apparatus of claim 18, wherein the at least one threshold parameter comprises an A1C value, and the threshold value is set to a predetermined reduction in the A1C value over a specified period.

21. The apparatus of claim 18, wherein the at least one threshold parameter comprises a duration of use of the automated medicament delivery system, and the threshold value is set to a predetermined number of weeks of continuous use.

22. The apparatus of claim 18, wherein the at least one processor is configured to evaluate a plurality of threshold parameters using a weighted combination, and unlock the capability based on a combined value exceeding a predetermined threshold.

23. The apparatus of claim 18, comprising a helper artificial intelligence module configured to provide interactive training materials to the user upon unlocking access to training features.

24. A non-transitory computer-readable medium storing instructions that, when executed by at least one processor, cause the at least one processor to: operate an automated medicament delivery system in a default mode, wherein at least one operating parameter is preset for safe medicament delivery; monitor at least one threshold parameter; and unlock a user capability to manually adjust the at least one operating parameter upon the at least one threshold parameter meeting a threshold value.

25. The non-transitory computer-readable medium of claim 24, wherein the instructions cause the at least one processor to generate an alert to notify the user of the unlocking option.

26. The non-transitory computer-readable medium of claim 24, wherein the instructions cause the at least one processor to revert to the default mode if the threshold value is no longer met, requiring approval from a healthcare professional to resume adjusted operations.

27. A system for automated medicament delivery, comprising: an analyte sensor configured to obtain analyte data from a user; a delivery unit configured to administer medicament based on the analyte data; a controller comprising adaptive control logic configured to: initialize the system in a default mode with preset operating parameters; evaluate evidence of improved user experience or outcomes based on at least one threshold parameter; and progressively unlock features of the system, including meal announcement capabilities and customizable basal profiles, as the evidence meets predefined conditions.

28. The system of claim 27, comprising a handheld electronic computing device communicatively coupled to the controller, wherein the unlocked features are accessible via a user interface on the handheld electronic computing device.

29. The system of claim 27, wherein the adaptive control logic is configured to use a condition processing chain that weights multiple inputs corresponding to different threshold parameters and compares a combined value to an adjustable threshold.

30. The system of claim 27, wherein the unlocked features include access to a helper artificial intelligence for providing real-time training support, the helper artificial intelligence trained on instructional materials including user manuals and videos.
